(19) United States
(12) Patent Application Publication
(10) Pub. No.: US 2024/0369561 A1
(43) Pub. Date: Nov. 7, 2024

(54) ANTIGEN CD111 AS A NEW SPECIFIC DIAGNOSTIC AND THERAPEUTIC MARKER FOR POLYMORPHONUCLEAR MYELOID-DERIVED SUPPRESSOR CELLS (PMN-MDSCS)

(71) Applicant: OSPEDALE PEDIATRICO BAMBINO GESU', Roma (IT)

(72) Inventors: Lorenzo MORETTA, Roma (IT); Paola VACCA, Roma (IT); Nicola TUMINO, Roma (IT)

(21) Appl. No.: 18/293,535

(22) PCT Filed: Aug. 1, 2022
(86) PCT No.: PCT/IT2022/050220

§ 371 Date: Jan. 30, 2024

(30) Foreign Application Priority Data
Aug. 2, 2021 (IT) 102021000020702

(51) Int. Cl. G01N 33/574; A61K 39/00; C07K 16/28

(52) U.S. Cl. CPC G01N 33/57484; C07K 16/2803; G01N 33/57426; A61K 2039/505; G01N 2333/70596

(57) ABSTRACT

A method for detecting polymorphonuclear myeloid-derived suppressor cells (PMN-MDSCs) in a biological sample employs the CD111 antigen as a marker for said cells and the use of said marker as an immunotherapeutic target. The CD111 antigen serves as a specific diagnostic and therapeutic marker for polymorphonuclear myeloid-derived suppressor cells (PMN-MDSCs).

The present invention relates to the CD111 antigen as a new specific diagnostic and therapeutic marker for polymorphonuclear myeloid-derived suppressor cells (PMN-MDSCs).

In particular, the invention relates to a method for detecting polymorphonuclear myeloid-derived suppressor cells (PMN-MDSCs) in a biological sample by employing the CD111 antigen as a marker for said cells and the use of said marker as an immunotherapeutic target.

It is known that myeloid-derived suppressor cells (MDSCs) comprise a heterogeneous population of mature and immature myeloid cells with immunoregulatory activity. MDSCs originate from common haematopoietic progenitor cells and are capable of suppressing both the innate immune system and the adaptive immune system of an individual.

In healthy individuals, the precursors of the myeloid line (IMCs) are about 2.5% of peripheral blood mononuclear cells (PBMCs), are generated in bone marrow and differentiate into granulocytes, macrophages or dendritic cells. Conversely, pathological conditions such as cancer, various infectious diseases, sepsis, traumas, bone marrow transplantation and some autoimmune diseases induce a partial block of IMC differentiation and a subsequent accumulation of MDSCs. Substantial evidence suggests the importance of MDSCs in human neoplasms, as they are capable of favouring tumour progression, metastasis and the induction of immune system tolerance towards the tumour (1,2). In particular, the study of these cells has shown that their expansion is correlated to various pathological conditions characterised by an inflammatory state, including infections, tumours and autoimmune diseases. Furthermore, it has been highlighted that certain factors may promote both the expansion and activation of MDSCs. Most evidence available to date in the literature is mainly based on the study of animal tumour models and cancer patients. However, it is very important to study these cells also in autoimmune pathologies, in which a hyperactivated immune system must be regulated. In these autoimmune pathologies, MDSCs have demonstrated to be an advantage for maintaining the homeostasis of the immune response (3).

Due to the phenotypic and functional heterogeneity of MDSCs, the exact mechanism whereby they develop, accumulate and carry out their activity has not yet been clearly defined. The presence and inhibitory effect of PMN-MDSCs has previously been demonstrated in vitro in the context of haploidentical transplantation for the treatment of high-risk leukaemia. In particular, in the context of haematopoietic stem cell transplantation, the mature NK cells of the donor play an important role in eliminating the residual leukaemic cells in the patient after the regime of conditioning with chemo/radiotherapy. However, in the context of “αβ T- and B-depleted” transplantation, the anti-leukaemic effect of the donor's NK cells is greatly inhibited. As previously demonstrated, in the graft of donors routinely “mobilised” with G-CSF, in addition to a significant increase in haematopoietic stem cells, there is an accumulation of PMN-MDSCs. These cells inhibit the activity of the donor's mature NK cells with different mechanisms (IDO, PGE2 and the release of exosomes), thus compromising the anti-leukaemic effect of the donor's mature NK cells (4).

In patients with tumours, moreover, a correlation has been observed between the presence of PMN-MDSCs in peripheral blood and the response to therapy (1).

Therefore, PMN-MDSCs show an extraordinary ability to inhibit the anti-tumour response of immune cells. In view of the immunosuppressant role of PMN-MDSCs in different pathologies, it would thus be important to be able to easily identify them by means of a marker capable of recognising them directly, in order to be able to eliminate them, with a targeted selection, both from the aphaeretic product, in the context of stem cell transplantation, and in the peripheral blood of patients with neoplasia. In particular, in the context of transplantation, the direct identification and selection/elimination of these cells would enable a functional recovery of the anti-leukaemic activity (GvL) of NK cells, whilst in patients with neoplasia it would make it possible to easily verify their presence in peripheral blood and to evaluate both the effectiveness of the therapy and any relapse of disease.

However, no exclusive marker for identifying PMN-MDSCs has yet been identified to date.

At present, in order to identify MDSCs it is necessary to combine various markers that allow the MDSCs to be divided into two groups: monocytic (MO) and polymorphonuclear (PMN). Since both groups of MDSCs are morphologically similar to mature myeloid cells and neutrophils, in order to identify them it is necessary to evaluate both the expression of particular surface markers in combination with one another, and their immunosuppressant activity, as demonstrated in previous studies (5). MDSCs are identified as Lineage (CD3/56/19/123) HLA-DR^{low/neg}CD33⁺CD11b⁺; MO-MDSCs are CD14⁺, whereas PMN-MDSCs are CD15⁺CD66b⁺.

For the identification of these cells it is necessary to carry out procedures of layering over a density gradient medium followed by a complex phenotype analysis which determines the expression or absence of different markers and, finally, an evaluation of the immunosuppressant potential by means of functional tests.

In the light of the above, there clearly appears to be a need to provide new markers for the selective identification of PMN-MDSCs which overcome the disadvantages of the known markers.

The solution according to the present invention, which aims to provides a specific marker for identifying PMN-MDSCs, fits into this context.

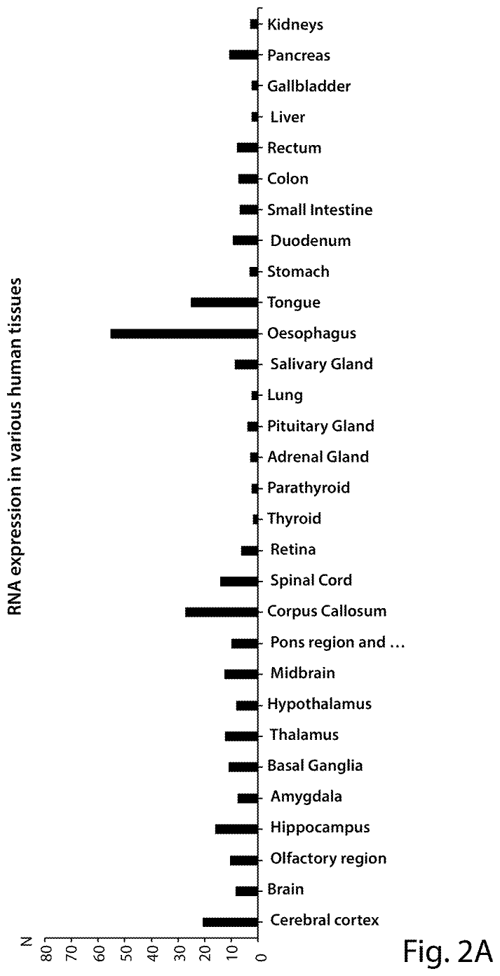

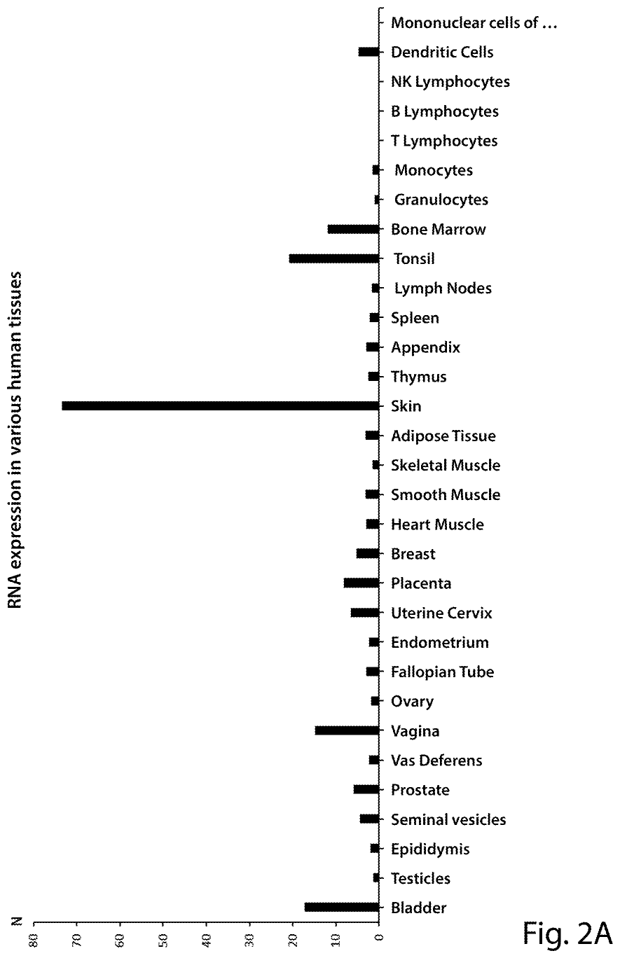

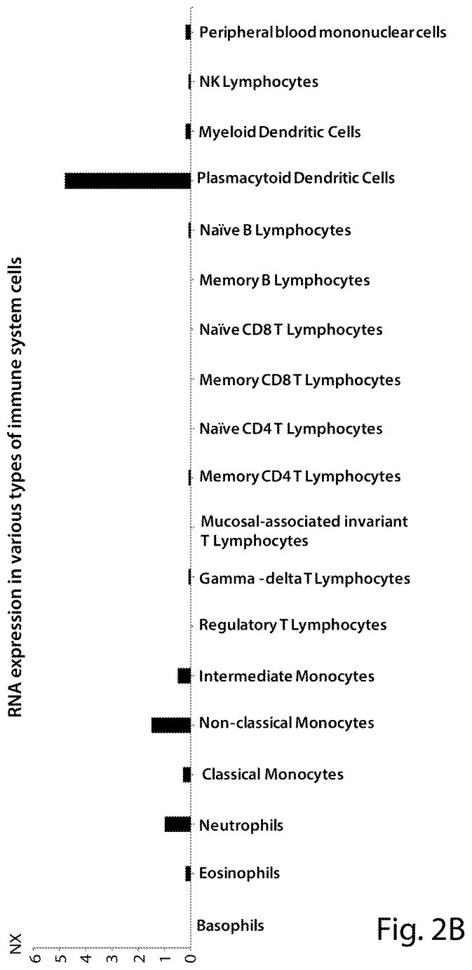

It is well known that the CD111 marker, also known as Poliovirus receptor-related 1 (PVRL1) or nectin-1, is a human protein belonging to the superfamily of immunoglobulins and also considered a member of the nectins. It is a membrane protein with three extracellular immunoglobulin domains, a single transmembrane helix and a cytoplasmic tail. CD111 is an adhesion molecule that functions in a Ca²⁺-independent manner. As also confirmed by the experimental data shown further below, in particular in FIG. 2A, the CD111 marker is localised in the junctions of various tissues (FIG. 2A), in particular between the junctions of epithelial tissue or in the chemical synapses of neurons. In the chemical synapse PVRL1 interacts with PVRL3 (nectin-3) and both proteins can be found in neuronal tissue already in the early stages of brain development. The CD111 protein also plays a role in the entry into cells of the Herpes simplex virus by interacting with the viral glycoprotein D (gD) (6).

According to the present invention, it has now been found that the CD111 marker enables PMN-MDSCs to be selectively identified. In greater detail, as shown by the experimental data reported further below, according to the present invention it has been observed that all PMN-MDSCs identified by means of known methods express the CD111 marker. It is known that PMN-MDSCs are highly abundant in the peripheral blood of individuals with tumours compared to healthy individuals.

Unlike the known methods, the CD111 marker according to the present invention is advantageously capable of distinguishing PMN-MDSCs from their neutrophilic counterpart without there being any need to separate the mononuclear cells. In fact, in the absence of a step of layering the peripheral blood, aimed at the purification of PBMCs, PMN-MDSCs and neutrophils show to have the same morphology when analysed by both microscopy and flow cytometry. Therefore, direct identification of these cells in peripheral blood according to the known methods to date is impossible. Surprisingly, through the identification of the CD111 marker according to the present invention, PMN-MDSCs can be selectively identified directly in peripheral blood, as neutrophils do not express this marker.

Therefore, the evaluation of CD111 expression according to the present invention advantageously allows for a rapid, direct identification of PMN-MDSCs directly in peripheral blood samples and the possibility of studying their impact on the disease course and the response to the various therapeutic approaches used. As mentioned above, it is known, in fact, that PMN-MDSCs drastically inhibit the functionality of the lymphoid effector cells of the immune system, such as NK cells and T lymphocytes.

Furthermore, the CD111 marker according to the present invention can be advantageously employed as an immunotherapeutic target/marker in order to eliminate this cell subset, i.e. PMN-MDSCs, thereby restoring the antitumour functions of the effector cells of the immune system.

It is therefore a specific object of the present invention the use of the CD111 protein (gene ID: 5818) as a marker for detecting polymorphonuclear myeloid-derived suppressor cells (PMN-MDSC) in a biological sample.

According to the present invention, the biological sample can be selected from a liquid sample, such as, for example, peripheral blood, ascites, synovial fluids, or a solid tissue such as, for example, fresh, paraffin embedded or cryopreserved tissue biopsies.

The present invention further relates to a method for detecting polymorphonuclear myeloid-derived suppressor cells (PMN-MDSC) in a biological sample, said method comprising detecting said cells through identification of the CD111 protein as a marker for said cells. According to the present invention, the biological sample can be selected from a liquid sample such as, for example, peripheral blood, ascites, synovial fluids, or a solid tissue such as, for example, fresh, paraffin embedded or cryopreserved tissue biopsies. The method according to the present invention can be conducted by flow cytometry or immunohistochemical analysis.

According to the present invention, the CD111 protein can be advantageously employed as an immunotherapeutic target. For example, the CD111 marker can be employed to prepare products capable of inhibiting and/or selectively eliminating polymorphonuclear myeloid-derived suppressor cells, said products having the CD111 marker as their target. The selective elimination of these cells enables a restoration of the antitumour functions of the effector cells of the immune system.

The present invention further relates to an inhibitor product of the polymorphonuclear myeloid-derived suppressor cells, for use in the treatment of tumours or infectious diseases, said inhibitor product having the CD111 marker as its target.

The inhibitor product according to the present invention, therefore, is a molecule having the CD111 molecule as its specific target and which, by binding thereto, is capable of recognising and eliminating/inhibiting PMN-MDSCs. In particular, the binding of said inhibitor product with CD111 is able to mediate the blocking of the suppressant functions of PMN-MDSCs or of inducing the death of the cells themselves (PMN-MDSCs).

According to the present invention, said inhibitor product can be selected from an anti-CD111 monoclonal antibody or an effector cell (e.g. T and NK lymphocytes) genetically modified with a construct, in particular a receptor, capable of selectively recognising the CD111 protein.

In particular, when the inhibitor product employed in the treatment of tumours or infectious diseases according to the invention is a monoclonal antibody capable of selectively recognising CD111, said antibody has the advantage of being able to be employed according to two approaches. According to a first approach, said antibody according to the invention can be administered by itself for the purpose of activating the endogenous immune system: the constant portion of the immunoglobulin (Fc) of the antibody can be recognised by endogenous immune system cells which express the receptors for the Fc portion (natural killer cells and monocytes). This specific recognition ensures the activation of the process identified as antibody-dependent cell-mediated cytotoxicity (ADCC). Furthermore, the Fc of the antibody can be recognised by the complement system and induce the opsonisation and elimination of CD111-positive cells.

According to a further therapeutic approach according to the invention, the Fc fragment of said antibody can be bound to substances, such as, for example, toxins, radioisotopes or drugs, which are capable of specifically inducing the death of myeloid-derived suppressor cells of the CD111-positive polymorphonuclear type. In this manner one would advantageously combine the binding specificity of the anti-CD111 antibody with the pharmacological activity of said substances. Therefore, according to the present invention, said inhibitor product can be an anti-CD111 monoclonal antibody, wherein said antibody, or, more in particular the Fc fragment of said antibody, is bound to a substance capable of inducing the death of myeloid-derived suppressor cells of the polymorphonuclear type, such as, for example, a drug, a toxin or a radioisotope.

According to the present invention, the tumour to be treated can be selected from solid tumours, for example lung cancer and neuroblastoma, or a paediatric or adult blood cancer, for example leukaemia.

Furthermore, the present invention relates to a process for preparing a product for the transplantation of stem cells, said process comprising a step of eliminating the polymorphonuclear myeloid-derived suppressor cells in an aphaeretic sample comprising stem cells by employing an anti-CD111 monoclonal antibody.

The present invention will now be described by way of illustration but not limitation, according to a preferred embodiment thereof, with particular reference to the examples and figures of the appended drawings, in which:

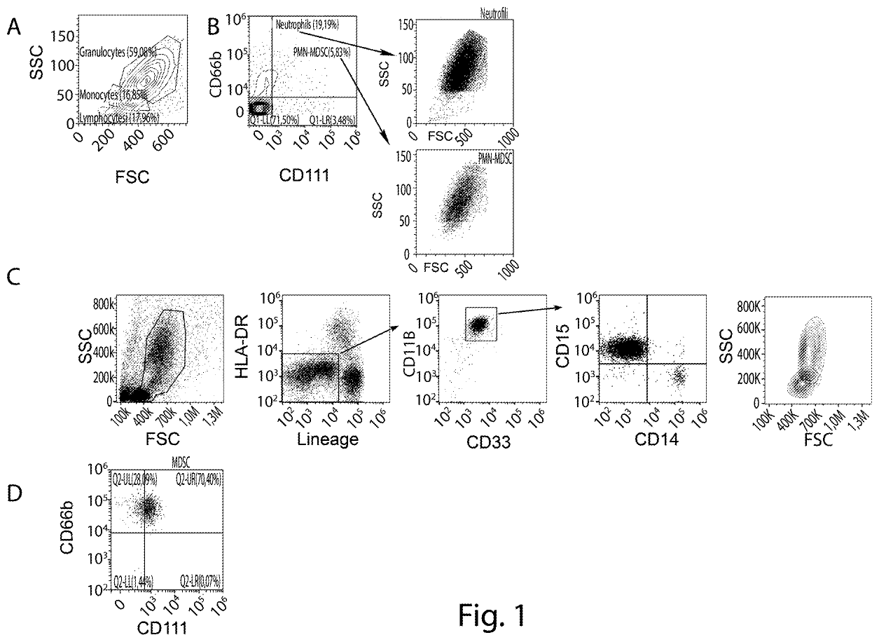

FIG. 1 shows (A) identification of different leukocyte subpopulations by flow cytometry analysis, evaluation of peripheral blood morphology (FSC) and granularity (SSC); (B) expression of CD66b and CD111; (C) identification of PMN-MDSCs in isolated PBMCs; (D) expression of CD111 in the PMN-MDSCs identified;

FIG. 2 shows (A) expression of CD111-encoding mRNA in various human tissues using the protein atlas dataset; (B) expression of CD111-encoding mRNA in various human cell subsets of the immune system using the protein atlas dataset;

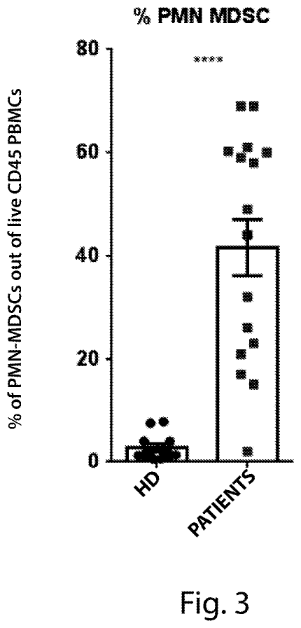

FIG. 3 shows the frequency of PMN-MDSCs in the PBMCs derived from peripheral blood of healthy donors and patients with lung cancer (Patients).

### EXAMPLE 1: STUDY OF THE CD111 PROTEIN AS A MARKER FOR PMN-MDSCS

### Materials and Methods

### Purification, Isolation and Cell Cultures:

Declaration pursuant to Art. 170 bis of the Industrial Property Code (CPI): all the biological materials of human origin used in the experiment were collected and used with the express free and informed consent to such collection and use of the person from whom that material was collected, on the basis of current legislation. (Ethics Committee: 1. Bambino Gesù Paediatric Hospital Ref. No. 132 28/01/2019; 2. Local Health Authority 3 (ASL3, Genoa) code: N9-13,2013; ID: 4975,2020).

The aphaeretic product or peripheral blood was layered over a density gradient medium (Ficoll) after being diluted with an equal volume (1:1 ratio) of phosphate-buffered saline (PBS). The blood thus diluted was slowly layered over Ficoll-Paque, maintaining a 2:1 blood: ficoll volume ratio. The layered sample was then centrifuged in a centrifuge with a tilting rotor at 400×g for 20 min at 20° C.; the deceleration ramp was set at minimum or deactivated. At the end of centrifugation, the samples were gently removed from the centrifuge to prevent mixing of the phases. For the purification of lymphocytes and MDSCs, ⅔ of the upper layer (containing plasma and platelets) was cautiously aspirated until the interphase (containing the mononuclear cells) was near; then the entire layer of lymphocytes and MDSCs was aspirated and transferred into a new test tube. Three volumes of phosphate-buffered saline were then added to the layer of cells collected and centrifuged at 100×g 20° C. for 10 min and the supernatants were eliminated. This washing was repeated 2 times. Finally, the cell pellet was once again suspended in a medium suitable for the applications. Subsequently, a cell count was performed and viability was evaluated by diluting the cells in Trypan blue in a 1:1 ratio and then performing a cell count using an automated cell counter.

### Flow Cytometry Analysis

For the identification of the MDSCs, a multiparametric panel was designed using a mix of monoclonal antibodies specific for different surface markers. The antibodies used were: CD11b-FITC, HLADR-PE, CD14-ECD, CD33-PC7, CD66-APC, CD3-APC-A700, CD19-APC-A700, CD45-KrO, CD56-BV650 and CD123-APC-A700. 100 μl of cell suspension with a concentration of 5 million/ml are necessary for marking. The mix of antibodies was added to the cells. After 20 minutes of incubation at 4° C., a washing in PBS was carried out and the expression of the markers was evaluated by flow cytometry (Cytoflex LX of Beckman Coulter). The data obtained were subsequently analysed using FlowJO software.

Analysis of the Expression of CD111-Encoding mRNA by Means of the Protein Atlas

An evaluation of CD111 expression was carried out using the dataset available online at the address https://www.proteinatlas.org/. The analysis was performed by evaluating expression in different human tissues and in various cell subsets of the immune system. Use of the Human Protein Atlas is licenced internationally through Creative Commons Attribution-ShareAlike 3.0, for all parts of the database protected by copyright.

### Results and Discussion

### The CD111 Marker Identifies PMN-MDSCs in an Exclusive Manner

MDSCs are morphologically similar to mature myeloid cells and neutrophils; therefore, in order to identify them it is necessary to evaluate the expression of various surface markers in combination with one another. As mentioned above, previous studies have demonstrated that MDSCs are identified as: Lineage HLA-DR^{low/neg}CD33⁺CD11b⁺; MO-MDSCs are CD14⁺ whereas PMN-MDSCs are CD15⁺CD66b⁺ (7).

Peripheral blood (PB) of healthy donors was analysed to evaluate the presence of PMN-MDSCs. As was demonstrated, at present the identification of PMN-MDSCs requires separation of the peripheral blood cells (PBMC) by means of a density gradient. In fact, in the peripheral blood of healthy donors different cell populations can be discriminated on the basis of cell granularity (SSC) and dimensions (FSC). As shown in FIG. 1A, granulocytes are characterised by high SSC and FSC values, monocytes have intermediate SSC and FSC values whereas lymphocytes, which include T, B and NK lymphocytes, represent the population with the lowest SSC and FSC. However, PMN-MDSCs also have a morphology comparable to that of neutrophils. FIG. 1B shows that a part of the CD66b+ cells (neutrophils) include a small population that co-expresses CD111. If one goes on to evaluate the FSC and SSC (FIG. 1B) of the two subpopulations (CD66b⁺/CD111⁻ and CD66b⁺/CD111⁺) it can be deduced that both show the same morphology.

The blood was subsequently layered over a density gradient medium (Ficoll) in order to separate the peripheral blood mononuclear cells (PBMCs) from neutrophils and red blood cells. The mononuclear cells that formed after layering were analysed for the presence of PMN-MDSCs. Multiparametric flow cytometry was used for the purpose of identifying these cells in the sample of mononuclear cells. As shown in FIG. 1C a sequential gating strategy is necessary in order to be able to confirm that the cells belong to the PMN-MDSC subpopulation. In particular, PMN-MDSCs do not express the markers (CD3/56/19/123) for the lymphocyte line (lin−) and they express low or negative levels of HLA-DR, but show to be CD33⁺ and CD11b⁺. Within this subpopulation PMN-MDSCs express CD66b and CD15. Finally, when CD111 expression within the PMN-MDSCs identified was evaluated, it was observed that all these cells express that marker in a homogeneous manner (FIG. 1D). Therefore, since in the absence of the step of layering over a density gradient PMN-MDSCs and neutrophils appear to have the same morphology when analysed both by microscopy and flow cytometry, the direct identification of these cells in peripheral blood has been impossible to date. On the basis of what was observed (FIG. 1), it can be deduced that CD111 expression in peripheral blood makes it possible to distinguish PMN-MDSCs from their neutrophilic counterpart without there being a need to separate the mononuclear cells.

### Evaluation of CD111 Expression in Various Tissue Regions

With the aim of confirming that the expression of the CD111 antigen was limited to the population of PMN-MDSCs present in peripheral blood under physiological and pathological conditions, an analysis of CD111 expression was performed using a database available free of charge online which provides information regarding mRNA expression levels. The data analysed demonstrate that plasmacytoid dendritic cells are the only ones that express CD111-encoding mRNA (FIG. 2B). However, these cells represent less than 1% of the circulating cells of peripheral blood. Furthermore, the expression of this CD111-encoding mRNA in these cells is very low and the presence of the mRNA is not direct evidence of protein expression. It is worth considering that within this database, which contains data regarding the majority of peripheral blood cell subpopulations, PMN-MDSCs have not been analysed. In particular, as regards the myeloid category, only dendritic cells, monocytes and neutrophils are analysed.

### CD111 as a New Specific Diagnostic and Therapeutic Marker for PMN-MDSCs

The presence of PMN-MDSCs was analysed in different peripheral blood samples drawn from healthy donors and patients with a tumour. As was demonstrated, in healthy donors the frequency of PMN-MDSCs is very low and in the range of 0% to 5% of peripheral blood. This frequency increases considerably after treatment with the G-CSF used to mobilise the haematopoietic stem cells that will subsequently be infused into patients affected by haematological neoplasia (4). Moreover, in patients affected by pulmonary neoplasia one observes an increase in PMN-MDSCs compared to the average frequency observed blood in healthy donors (FIG. 3).

The evaluation of CD111 expression enables a rapid and direct identification of PMN-MDSCs to be achieved directly in peripheral blood samples, making it possible to study the impact thereof on the disease course and the response to the different therapeutic approaches used. It is known, in fact, that PMN-MDSCs drastically inhibit the functionality of the lymphoid effector cells of the immune system, such as NK cells and T lymphocytes. Furthermore, it is possible to employ CD111 as an immunotherapeutic target in order to eliminate this cell subset, thereby restoring the antitumour functions of the effector cells of the immune system.

### REFERENCES

1. Kumar V, Patel S, Tcyganov E, Gabrilovich D I. The Nature of Myeloid-Derived Suppressor Cells in the Tumor Microenvironment. Trends in immunology 2016; 37 (3): 208-20 doi 10.1016/j.it.2016.01.004.
2. Nagaraj S, Youn J I, Gabrilovich D I. Reciprocal relationship between myeloid-derived suppressor cells and T cells. Journal of immunology 2013; 191 (1): 17-23 doi 10.4049/jimmunol.1300654.
3. Pawelec G, Verschoor C P, Ostrand-Rosenberg S. Myeloid-Derived Suppressor Cells: Not Only in Tumor Immunity. Frontiers in immunology 2019; 10:1099 doi 10.3389/fimmu.2019.01099.
4. Tumino N, Besi F, Di Pace A L, Mariotti F R, Merli P, Li Pira G, et al. PMN-MDSC are a new target to rescue graft-versus-leukemia activity of NK cells in haplo-HSC transplantation. Leukemia 2020; 34 (3): 932-7 doi 10.1038/s41375-019-0585-7.
5. Bronte V, Brandau S, Chen S H, Colombo M P, Frey A B, Greten T F, et al. Recommendations for myeloid-derived suppressor cell nomenclature and characterization standards. Nature communications 2016; 7:12150 doi 10.1038/ncomms12150.
6. Di Giovine P, Settembre E C, Bhargava A K, Luftig M A, Lou H, Cohen G H, et al. Structure of herpes simplex virus glycoprotein D bound to the human receptor nectin-1. PLOS pathogens 2011; 7 (9): e1002277 doi 10.1371/journal.ppat.1002277.

1-2. (canceled)

3. A method for detecting polymorphonuclear myeloid-derived suppressor cells, in a biological sample, said method comprising detecting said cells through the identification of CD111 protein as a marker for said cells.

4. The method according to claim 3, wherein the biological sample comprises a liquid sample or a solid tissue.

5. The method according to claim 3, said method being carried out by flow cytometry or immunohistochemical analysis.

6. A method for treating a tumor or an infectious disease in a subject in need thereof, the method comprising administering an inhibitor product of polymorphonuclear myeloid-derived suppressor cells, said inhibitor product having CD111 protein as its target.

7. The method according to claim 6, wherein said inhibitor product comprises an anti-CD111 monoclonal antibody or an effector cell that is genetically modified with a construct so as to selectively recognize the CD111 protein.

8. The method according to claim 7, wherein said inhibitor product is an anti-CD111 monoclonal antibody, and wherein said antibody is bound to a substance capable of inducing death of myeloid-derived suppressor cells of the polymorphonuclear type.

9. The method according to claim 6, wherein the tumour is a solid tumor or pediatric or adult blood cancer.

10. A process for preparing a product for stem cell transplantation, said process comprising eliminating polymorphonuclear myeloid-derived suppressor cells in an apheretic sample comprising stem cells by employing an anti-CD111 monoclonal antibody.

11. The method according to claim 4, wherein the liquid sample comprises peripheral blood, ascites, or synovial fluid.

12. The method according to claim 4, wherein the solid tissue comprises fresh, paraffin-embedded or cryopreserved tissue biopsies.

13. The method according to claim 8, wherein the substance comprises a drug, a toxin, or a radioisotope.

14. The method according to claim 9, wherein the solid tumor is lung cancer or neuroblastoma.

15. The method according to claim 9, wherein the pediatric or adult blood cancer is leukemia.
